(19) United States
(12) Patent Application Publication
(10) Pub. No.: US 2019/0038650 A1
(43) Pub. Date: Feb. 7, 2019

(54) COMPOSITIONS AND METHODS FOR THE TREATMENT OF ATHEROSCLEROSIS AND HEPATOSTEATOSIS AND OTHER DISEASES

(71) Applicants: Babak Razani, St. Louis, MO (US); Abhinav Diwan, St. Louis, MO (US)

(72) Inventors: Babak Razani, St. Louis, MO (US); Abhinav Diwan, St. Louis, MO (US)

(21) Appl. No.: 16/074,023

(22) PCT Filed: Feb. 1, 2017
(86) PCT No.: PCT/US2017/016064

§ 371 Date: Jul. 30, 2018

Related U.S. Application Data
(60) Provisional application No. 62/289,604, filed on Feb. 1, 2016.

(51) Int. Cl. A61K 31/7016; A61K 9/00; A61P 9/10; A61P 3/10; A61P 3/04; A61P 1/16

(52) U.S. Cl. CPC A61K 31/7016; A61K 9/0019; A61K 9/0053; A61P 9/10; A61K 31/7034; A61P 3/04; A61P 1/16; A61K 2300/00; A61P 3/10

(57) ABSTRACT

The present disclosure provides compositions comprising trehalose, and optionally a trehalase inhibitor, for the treatment of atherosclerosis and liver steatosis.

### CROSS REFERENCE TO RELATED APPLICATIONS

This application claims the benefit of U.S. Provisional Application No. 62/289,604, filed Feb. 1, 2016, the disclosure of which is hereby incorporated by reference in its entirety.

### FIELD OF THE INVENTION

The present disclosure provides compositions comprising trehalose, and optionally a trehalase inhibitor, for the treatment of atherosclerosis and hepatosteatosis.

### BACKGROUND OF THE INVENTION

Atherosclerotic cardiovascular disease (ACD) is the leading cause of mortality worldwide. Atherosclerosis is an inflammatory disease of the arteries associated with lipid and other metabolic alterations and is the major cause of cardiovascular diseases. Atherosclerotic cardiovascular disease (ACD) includes two major conditions: ischemic heart disease (IHD) and cerebrovascular disease (mainly ischemic stroke). IHD and stroke are the world's first and third causes of death, respectively, causing 247.9 deaths/100,000 persons in 2013, representing 84.5% of cardiovascular deaths and 28.2% of all-cause mortality. Other less prevalent complications of atherosclerosis include atherosclerosis of the aorta and peripheral vascular disease. In 2011, coronary atherosclerosis was one of the top ten most expensive conditions seen during inpatient hospitalizations in the U.S., with aggregate inpatient hospital costs of $10.4 billion. Given the high incidence and substantial economic burden of atherosclerosis, new methods to treat and prevent atherosclerosis are needed.

Additionally, cardiovascular disease is a major cause of death in patients on dialysis. The severity of renal dysfunction correlates with the severity of increased cardiovascular risk. Accordingly, patients on dialysis are subject to higher rate of atherosclerosis that contributes to higher mortality. Thus, there is a need in the art for treatment that specifically targets this group with high unmet need.

Hepatosteatosis, also known as fatty liver or fatty liver disease (FLD), is a reversible condition wherein large vacuoles of triglyceride fat accumulate in liver cells via the process of steatosis (i.e., abnormal retention of lipids within a cell). Despite having multiple causes, fatty liver can be considered a single disease that occurs worldwide in those with excessive alcohol intake and the obese (with or without effects of insulin resistance). The condition is also associated with other diseases that influence fat metabolism. When this process of fat metabolism is disrupted, the fat can accumulate in the liver in excessive amounts, thus resulting in a fatty liver. The prevalence of hepatosteatosis in the general population ranges from 10% to 24% in various countries. Hepatosteatosis is the most common cause of abnormal liver function tests in the United States. Fatty livers occur in 33% of European-Americans, 45% of Hispanic-Americans, and 24% of African-Americans. Thus, there is a need in the art for a treatment that targets heptatosteatosis, as well as atherosclerosis.

### SUMMARY OF THE INVENTION

In an aspect, the disclosure provides a method for treating atherosclerosis in a subject in need thereof. The method comprises administering a therapeutically effective amount of a composition comprising trehalose, and optionally a trehalase inhibitor, to the subject.

In another aspect, the disclosure provides a method for treating liver steatosis in a subject in need thereof. The method comprises administering a therapeutically effective amount of a composition comprising trehalose, and optionally a trehalase inhibitor, to the subject.

In still another aspect, the disclosure provides a method for treating atherosclerosis and liver steatosis, simultaneously, in a subject in need thereof. The method comprises administering a therapeutically effective amount of a composition comprising trehalose, and optionally a trehalase inhibitor, to the subject.

In still yet another aspect, the disclosure provides a method for treating atherosclerosis in a subject undergoing dialysis. The method comprises administering a therapeutically effective amount of a composition comprising trehalose, and optionally a trehalase inhibitor, to the subject.

In any of the foregoing aspects, the composition may be administered orally or intravenously. In an embodiment, when the composition is administered orally, the trehalase inhibitor is present. In another embodiment, when the composition is administered intravenously, the trehalase inhibitor is absent. The trehalase inhibitor may be validamycin. In certain embodiments, the composition is administered following dialysis. The composition may administered 3 times per week following dialysis. In an embodiment, the trehalase inhibitor is absent. In other embodiments, the composition is administered once weekly. The trehalose may be administered at a dose of 5 to 50 grams per administration. The trehalose may be administered at a dose of about 0.5 grams trehalose per kilogram body weight per administration. The trehalose may be administered at 8, 15 or 30 grams per day. The trehalose may be at 10% (w/v) of the composition. Treatment of liver steatosis may be measured by a reduction in fat mass or liver triglycerides. Treatment may be a reduction in the signs and symptoms associated with atherosclerosis and/or liver steatosis. In certain embodiments, insulin resistance, obesity and/or diabetes is also treated.

### BRIEF DESCRIPTION OF THE FIGURES

The application file contains at least one drawing executed in color. Copies of this patent application publication with color drawing(s) will be provided by the Office upon request and payment of the necessary fee.

FIG. 1 depicts a graph showing that administration of trehalose significantly reduces atherosclerotic lesion size relative to vehicle control.

FIG. 2A, FIG. 2B, FIG. 2C and FIG. 2D depict graphs showing that trehalose administration results in a significant reduction in adipose and liver fat. FIG. 2A depicts a graph showing that body mass is significantly reduced in animals administered trehalose. FIG. 2B depicts a graph showing that administration of trehalose significantly reduces fat mass but not lean mass. FIG. 2C depicts a graph showing that trehalose significantly reduces subcutaneous fat and liver fat. FIG. 2D depicts a graph showing that trehalose significantly reduced liver triglycerides.

FIG. 3 depicts a graph showing the results from an insulin tolerance test. The data shows less insulin resistance after trehalose administration as compared to saline and sucrose administration.

FIG. 4A and FIG. 4B depict graphs of control and trehalase knock out mice administered a trehalose tolerance test. The trehalose tolerance test was performed on n=3 non-targeted controls and n=5 chimeric mice by IP injecting 1 g/kg trehalose. FIG. 4A depicts the blood glucose levels at indicated times and FIG. 4B depicts the glucose area under curve (AUC). FIG. 4C depicts a graph showing that trehalase knockout mice exhibit significantly enhanced serum levels of trehalose.

FIG. 5 depicts a graph showing that co-administration of trehalose and the trehalase inhibitor, validamycin, significantly improves trehalose activity.

### DETAILED DESCRIPTION OF THE INVENTION

The present disclosure is based on the discovery that trehalose results in reduction in atherosclerotic plaque size and reduction in fatty liver deposits. Additionally, it was discovered that administration of trehalose with a trehalase inhibitor significantly improved its activity. Compositions and methods based on these findings are described in detail below.

### I. Compositions

The disclosure provides a composition comprising trehalose as the active ingredient. The disclosure also provides a composition comprising trehalose as the active ingredient and a trehalase inhibitor. In some embodiments, the composition comprises more than one trehalase inhibitor. “Trehalose” is a stable, nonreducing disaccharide with two glucose molecules linked in a 1,1 configuration. Like all disaccharides, trehalose is metabolized at the epithelial brush border to two D-glucose molecules. Less than 0.5% of ingested trehalose is absorbed into the blood stream where it is further metabolized by liver and kidney by trehalase. Oral trehalose in amounts exceeding 40-50 gram per day may cause diarrhea and bloating. Thus, in order to provide enhanced therapeutic amounts of trehalose in the cells, metabolism in the GI tract may be circumvented. Therefore, if the route of administration is oral, it is preferable to include a trehalase inhibitor in the composition. Stated another way, in the absence of a trehalase inhibitor, the preferred route of administration is parenteral. Accordingly, the inventors also developed a composition comprising trehalose and a trehalase inhibitor. The composition comprising trehalose as the active ingredient and a trehalase inhibitor is preferably administered orally. Additionally, a composition comprising trehalose, with or without a trehalase inhibitor, preferably comprises medical grade trehalose.

To date, the safety and toxicity of trehalose has been extensively investigated, and the substance was found to be safe when administered both orally and intravenously, in doses that are substantially higher than the intended therapeutic dose. The compositions of the current disclosure comprise, as an active agent, trehalose in a pharmaceutically acceptable form. The active agent, trehalose, may be administered in the form of the compound per se, as well as in the form of a salt, polymorph, ester, amide, prodrug, derivative, or the like, provided the salt, polymorph, ester, amide, prodrug or derivative is suitable pharmacologically. Salts, esters, amides, prodrugs and other derivatives of the active agents may be prepared using standard procedures known to those skilled in the art of synthetic organic chemistry and described for example, by J. March, Advanced Organic Chemistry: Reactions, Mechanisms and Structure, 4th Ed. (New York: Wiley-Interscience, 1992). For any active agents that may exist in enantiomeric forms, the active agent may be incorporated into the present compositions either as the racemate or in enantiomerically pure form.

In one embodiment, the purified trehalose is substantially free of contaminants resulting from the protein used in the enzymatic preparation process of the trehalose, such as organic solvents used in the process, e.g., ammonium, acetonitrile, acetamide, alcohol (e.g., methanol, ethanol, or isopropanol), TFA, ether or other contaminants. In this context “substantially” free of contaminants means that the contaminant content of the peptide at the end of the purification process is preferably less than 0.5%, less than 0.3%, less than 0.25%, less than 0.1%, less than 0.05%, less than 0.04%, less than 0.03%, less than 0.02%, less than 0.01%, less than 0.005%, less than 0.003%, or less than 0.001% of the total weight of the trehalose. The content of contaminants can be determined by conventional methods such as gas chromatography. Preferably, the residual solvents in the purified trehalose of the disclosure are less than the limits set in the ICH guidelines, e.g., IMPURITIES: GUIDELINE FOR RESIDUAL SOLVENTS Q3C(R5) (available at www.ich.org/fileadmin/Public_Web_Site/ICH_Products/Guidelines/Quality/Q3C/Step4/Q 3C_R5_Step4.pdf). For example, the purified trehalose contains <5000 ppm ethanol (e.g., <140 ppm), and/or <3000 ppm methanol.

Additionally, the composition contains less than 1.0, 0.9, 0.8, 0.75, 0.7, 0.6, 0.5, 0.4, 0.3, 0.2, 0.1 or less endotoxin units per mL.

The compositions of the current disclosure may also comprise a trehalase inhibitor in addition to trehalose. The composition may comprise 1, 2, 3, 4, or 5 trehalase inhibitors. Trehalase is a glycoside hydrolase enzyme located in the brush border of the small intestine that catalyzes the conversion of trehalose to glucose. Trehalases fall into the family GH37 of the Carbohydrate-Active Enzyme (CAZy) classification (EC 3.2.1.28). A compound that inhibits the enzymatic activity of trehalase may be used as a trehalase inhibitor. Non-limiting examples of trehalase inhibitors include validoxylamine A, validamycin A, trehazolin, 1-thiatrehazolin, suidatrestin, salbostatin, MDL 26537, casuarine-6-O-α-D-glucopyranoside, and the 86 kD protein from the american cockroach (Periplaneta americana) (See Hayakawa et al., J Biol Chem 1989; 264(27): 16165-16169, the disclosure of which is hereby incorporated by reference in its entirety). A trehalase inhibitor may also be those described in U.S. Pat. No. 5,354,685 and CN 101627763. Additional trehalase inhibitors may be determined by methods known in the art. For example, binding affinity of a compound to trehalase may be used to determine if the compound is an inhibitor for trehalase, wherein high affinity binding of the compound to trehalase indicates the compound is an inhibitor of trehalase. Further, enzymatic activity of trehalase in the presence of a compound may be used to determine if the compound is an inhibitor of trehalase, wherein a decrease in enzymatic activity indicates the compound is an inhibitor of trehalase. Additionally, a compound may be modeled onto the active site of trehalase to determine if the compound is an inhibitor of trehalase, wherein if the compound is modeled to have numerous interactions in the active site of trehalase, then the compound is a trehalase inhibitor. For example, see Gibson et al., Angew. Chem. Int. Ed 2007; 46: 4115-4119, the disclosure of which is hereby incorporated by reference in its entirety, which demonstrates the structure of trehalase and identifies methods of determining trehalase inhibitors.

The amount of trehalose, and optionally trehalase inhibitor, in the compositions disclosed herein will depend on a number of factors and will vary from subject to subject. Such factors include the severity of the symptoms, the patient's age, weight and general condition, and the judgment of the prescribing physician. Additionally, the activity of the trehalase inhibitor may be accounted for. Preferably an aqueous formulation is about 50%, 40%, 30%, 20%, 10%, 5% or less trehalose (w/v).

The present disclosure provides pharmaceutical compositions comprising trehalose, and optionally a trehalase inhibitor. The pharmaceutical composition comprises trehalose which is detailed above, as an active ingredient, and at least one pharmaceutically acceptable excipient. Additionally, the pharmaceutical composition comprises trehalose and a trehalase inhibitor, as active ingredients, and at least one pharmaceutically acceptable excipient.

The pharmaceutically acceptable excipient may be a diluent, a binder, a filler, a buffering agent, a pH modifying agent, a disintegrant, a dispersant, a preservative, a lubricant, taste-masking agent, a flavoring agent, or a coloring agent. The amount and types of excipients utilized to form pharmaceutical compositions may be selected according to known principles of pharmaceutical science.

In one embodiment, the excipient may be a diluent. The diluent may be compressible (i.e., plastically deformable) or abrasively brittle. Non-limiting examples of suitable compressible diluents include microcrystalline cellulose (MCC), cellulose derivatives, cellulose powder, cellulose esters (i.e., acetate and butyrate mixed esters), ethyl cellulose, methyl cellulose, hydroxypropyl cellulose, hydroxypropyl methylcellulose, sodium carboxymethylcellulose, corn starch, phosphated corn starch, pregelatinized corn starch, rice starch, potato starch, tapioca starch, starch-lactose, starch-calcium carbonate, sodium starch glycolate, glucose, fructose, lactose, lactose monohydrate, sucrose, xylose, lactitol, mannitol, malitol, sorbitol, xylitol, maltodextrin, and trehalose. Non-limiting examples of suitable abrasively brittle diluents include dibasic calcium phosphate (anhydrous or dihydrate), calcium phosphate tribasic, calcium carbonate, and magnesium carbonate.

In another embodiment, the excipient may be a binder. Suitable binders include, but are not limited to, starches, pregelatinized starches, gelatin, polyvinylpyrrolidone, cellulose, methylcellulose, sodium carboxymethylcellulose, ethylcellulose, polyacrylam ides, polyvinyloxoazolidone, polyvinylalcohols, C₁₂-C₁₈ fatty acid alcohol, polyethylene glycol, polyols, saccharides, oligosaccharides, polypeptides, oligopeptides, and combinations thereof.

In another embodiment, the excipient may be a filler. Suitable fillers include, but are not limited to, carbohydrates, inorganic compounds, and polyvinylpyrrolidone. By way of non-limiting example, the filler may be calcium sulfate, both di- and tri-basic, starch, calcium carbonate, magnesium carbonate, microcrystalline cellulose, dibasic calcium phosphate, magnesium carbonate, magnesium oxide, calcium silicate, talc, modified starches, lactose, sucrose, mannitol, or sorbitol.

In still another embodiment, the excipient may be a buffering agent. Representative examples of suitable buffering agents include, but are not limited to, phosphates, carbonates, citrates, tris buffers, and buffered saline salts (e.g., Tris buffered saline or phosphate buffered saline).

In various embodiments, the excipient may be a pH modifier. By way of non-limiting example, the pH modifying agent may be sodium carbonate, sodium bicarbonate, sodium citrate, citric acid, or phosphoric acid. Optimally the pH of the formulation is about 4.5 to 7.0. The osmolality of the formulation is about 280-330 mOsm/kg.

In a further embodiment, the excipient may be a disintegrant. The disintegrant may be non-effervescent or effervescent. Suitable examples of non-effervescent disintegrants include, but are not limited to, starches such as corn starch, potato starch, pregelatinized and modified starches thereof, sweeteners, clays, such as bentonite, micro-crystalline cellulose, alginates, sodium starch glycolate, gums such as agar, guar, locust bean, karaya, pecitin, and tragacanth. Non-limiting examples of suitable effervescent disintegrants include sodium bicarbonate in combination with citric acid and sodium bicarbonate in combination with tartaric acid.

In yet another embodiment, the excipient may be a dispersant or dispersing enhancing agent. Suitable dispersants may include, but are not limited to, starch, alginic acid, polyvinylpyrrolidones, guar gum, kaolin, bentonite, purified wood cellulose, sodium starch glycolate, isoamorphous silicate, and microcrystalline cellulose.

In another alternate embodiment, the excipient may be a preservative. Non-limiting examples of suitable preservatives include antioxidants, such as BHA, BHT, vitamin A, vitamin C, vitamin E, or retinyl palmitate, citric acid, sodium citrate; chelators such as EDTA or EGTA; and antimicrobials, such as parabens, chlorobutanol, or phenol.

In a further embodiment, the excipient may be a lubricant. Non-limiting examples of suitable lubricants include minerals such as talc or silica; and fats such as vegetable stearin, magnesium stearate or stearic acid.

In yet another embodiment, the excipient may be a taste-masking agent. Taste-masking materials include cellulose ethers; polyethylene glycols; polyvinyl alcohol; polyvinyl alcohol and polyethylene glycol copolymers; monoglycerides or triglycerides; acrylic polymers; mixtures of acrylic polymers with cellulose ethers; cellulose acetate phthalate; and combinations thereof.

In an alternate embodiment, the excipient may be a flavoring agent. Flavoring agents may be chosen from synthetic flavor oils and flavoring aromatics and/or natural oils, extracts from plants, leaves, flowers, fruits, and combinations thereof.

In still a further embodiment, the excipient may be a coloring agent. Suitable color additives include, but are not limited to, food, drug and cosmetic colors (FD&C), drug and cosmetic colors (D&C), or external drug and cosmetic colors (Ext. D&C).

The weight fraction of the excipient or combination of excipients in the composition may be about 99% or less, about 97% or less, about 95% or less, about 90% or less, about 85% or less, about 80% or less, about 75% or less, about 70% or less, about 65% or less, about 60% or less, about 55% or less, about 50% or less, about 45% or less, about 40% or less, about 35% or less, about 30% or less, about 25% or less, about 20% or less, about 15% or less, about 10% or less, about 5% or less, about 2%, or about 1% or less of the total weight of the composition.

The composition can be formulated into various dosage forms and administered by a number of different means that will deliver a therapeutically effective amount of the active ingredient. Such compositions can be administered orally or parenterally in dosage unit formulations containing conventional nontoxic pharmaceutically acceptable carriers, adjuvants, and vehicles as desired. The term parenteral as used herein includes subcutaneous, intravenous, intramuscular, or intrasternal injection, or infusion techniques. Formulation of drugs is discussed in, for example, Gennaro, A. R., Remington's Pharmaceutical Sciences, Mack Publishing Co., Easton, Pa. (18^{th} ed, 1995), and Liberman, H. A. and Lachman, L., Eds., Pharmaceutical Dosage Forms, Marcel Dekker Inc., New York, N.Y. (1980).

Solid dosage forms for oral administration include capsules, tablets, caplets, pills, powders, pellets, and granules. In such solid dosage forms, the active ingredient is ordinarily combined with one or more pharmaceutically acceptable excipients, examples of which are detailed above. Oral preparations may also be administered as aqueous suspensions, elixirs, or syrups. For these, the active ingredient may be combined with various sweetening or flavoring agents, coloring agents, and, if so desired, emulsifying and/or suspending agents, as well as diluents such as water, ethanol, glycerin, and combinations thereof.

For parenteral administration (including subcutaneous, intradermal, intravenous, intramuscular, and intraperitoneal), the preparation may be an aqueous or an oil-based solution. Aqueous solutions may include a sterile diluent such as water, saline solution, a pharmaceutically acceptable polyol such as glycerol, propylene glycol, or other synthetic solvents; an antibacterial and/or antifungal agent such as benzyl alcohol, methyl paraben, chlorobutanol, phenol, thimerosal, and the like; an antioxidant such as ascorbic acid or sodium bisulfite; a chelating agent such as etheylenediaminetetraacetic acid; a buffer such as acetate, citrate, or phosphate; and/or an agent for the adjustment of tonicity such as sodium chloride, dextrose, or a polyalcohol such as mannitol or sorbitol. The pH of the aqueous solution may be adjusted with acids or bases such as hydrochloric acid or sodium hydroxide. Oil-based solutions or suspensions may further comprise sesame, peanut, olive oil, or mineral oil.

In certain embodiments, an active ingredient of the disclosure is encapsulated in a suitable vehicle to either aid in the delivery of the compound to target cells, to increase the stability of the composition, or to minimize potential toxicity of the composition. As will be appreciated by a skilled artisan, a variety of vehicles are suitable for delivering a composition of the present disclosure. Non-limiting examples of suitable structured fluid delivery systems may include nanoparticles, liposomes, microemulsions, micelles, dendrimers and other phospholipid-containing systems. Methods of incorporating compositions into delivery vehicles are known in the art.

In one alternative embodiment, a liposome delivery vehicle may be utilized. Liposomes, depending upon the embodiment, are suitable for delivery of the active ingredients of the disclosure in view of their structural and chemical properties. Generally speaking, liposomes are spherical vesicles with a phospholipid bilayer membrane. The lipid bilayer of a liposome may fuse with other bilayers (e.g., the cell membrane), thus delivering the contents of the liposome to cells. In this manner, an active ingredient of the disclosure may be selectively delivered to a cell by encapsulation in a liposome that fuses with the targeted cell's membrane.

Liposomes may be comprised of a variety of different types of phospholipids having varying hydrocarbon chain lengths. Phospholipids generally comprise two fatty acids linked through glycerol phosphate to one of a variety of polar groups. Suitable phospholipids include phosphatidic acid (PA), phosphatidylserine (PS), phosphatidylinositol (PI), phosphatidylglycerol (PG), diphosphatidylglycerol (DPG), phosphatidylcholine (PC), and phosphatidylethanolamine (PE). The fatty acid chains comprising the phospholipids may range from about 6 to about 26 carbon atoms in length, and the lipid chains may be saturated or unsaturated. Suitable fatty acid chains include (common name presented in parentheses) n-dodecanoate (laurate), n-tetradecanoate (myristate), n-hexadecanoate (palmitate), n-octadecanoate (stearate), n-eicosanoate (arachidate), n-docosanoate (behenate), n-tetracosanoate (lignocerate), cis-9-hexadecenoate (palmitoleate), cis-9-octadecanoate (oleate), cis,cis-9,12-octadecandienoate (linoleate), all cis-9,12,15-octadecatrienoate (linolenate), and all cis-5,8,11,14-eicosatetraenoate (arachidonate). The two fatty acid chains of a phospholipid may be identical or different. Acceptable phospholipids include dioleoyl PS, dioleoyl PC, distearoyl PS, distearoyl PC, dimyristoyl PS, dimyristoyl PC, dipalmitoyl PG, stearoyl, oleoyl PS, palmitoyl, linolenyl PS, and the like.

The phospholipids may come from any natural source, and, as such, may comprise a mixture of phospholipids. For example, egg yolk is rich in PC, PG, and PE, soy beans contains PC, PE, PI, and PA, and animal brain or spinal cord is enriched in PS. Phospholipids may come from synthetic sources too. Mixtures of phospholipids having a varied ratio of individual phospholipids may be used. Mixtures of different phospholipids may result in liposome compositions having advantageous activity or stability of activity properties. The above mentioned phospholipids may be mixed, in optimal ratios with cationic lipids, such as N-(1-(2,3-dioleolyoxy)propyl)-N,N,N-trimethyl ammonium chloride, 1,1′-dioctadecyl-3,3,3′,3′-tetramethylindocarbocyanine perchloarate, 3,3′-deheptyloxacarbocyanine iodide, 1,1′-dedodecyl-3,3,3′,3′-tetramethylindocarbocyanine perchloarate, 1,1′-dioleyl-3,3,3′,3′-tetramethylindo carbocyanine methanesulfonate, N-4-(delinoleylaminostyryl)-N-methylpyridinium iodide, or 1,1,-dilinoleyl-3,3,3′,3′-tetramethylindocarbocyanine perchloarate.

Liposomes may optionally comprise sphingolipids, in which sphingosine is the structural counterpart of glycerol and one of the one fatty acids of a phosphoglyceride, or cholesterol, a major component of animal cell membranes. Liposomes may optionally, contain pegylated lipids, which are lipids covalently linked to polymers of polyethylene glycol (PEG). PEGs may range in size from about 500 to about 10,000 daltons.

Liposomes may further comprise a suitable solvent. The solvent may be an organic solvent or an inorganic solvent. Suitable solvents include, but are not limited to, dimethylsulfoxide (DMSO), methylpyrrolidone, N-methylpyrrolidone, acetronitrile, alcohols, dimethylformamide, tetrahydrofuran, or combinations thereof.

Liposomes carrying an active ingredient of the disclosure (i.e., having at least one methionine compound) may be prepared by any known method of preparing liposomes for drug delivery, such as, for example, detailed in U.S. Pat. Nos. 4,241,046, 4,394,448, 4,529,561, 4,755,388, 4,828,837, 4,925,661, 4,954,345, 4,957,735, 5,043,164, 5,064,655, 5,077,211 and 5,264,618, the disclosures of which are hereby incorporated by reference in their entirety. For example, liposomes may be prepared by sonicating lipids in an aqueous solution, solvent injection, lipid hydration, reverse evaporation, or freeze drying by repeated freezing and thawing. In a preferred embodiment the liposomes are formed by sonication. The liposomes may be multilamellar, which have many layers like an onion, or unilamellar. The liposomes may be large or small. Continued high-shear sonication tends to form smaller unilamellar liposomes.

As would be apparent to one of ordinary skill, all of the parameters that govern liposome formation may be varied. These parameters include, but are not limited to, temperature, pH, concentration of methionine compound, concentration and composition of lipid, concentration of multivalent cations, rate of mixing, presence of and concentration of solvent.

In another embodiment, an active ingredient of the disclosure may be delivered to a cell as a microemulsion. Microemulsions are generally clear, thermodynamically stable solutions comprising an aqueous solution, a surfactant, and “oil.” The “oil” in this case, is the supercritical fluid phase. The surfactant rests at the oil-water interface. Any of a variety of surfactants are suitable for use in microemulsion formulations including those described herein or otherwise known in the art. The aqueous microdomains suitable for use in the disclosure generally will have characteristic structural dimensions from about 5 nm to about 100 nm. Aggregates of this size are poor scatterers of visible light and hence, these solutions are optically clear. As will be appreciated by a skilled artisan, microemulsions can and will have a multitude of different microscopic structures including sphere, rod, or disc shaped aggregates. In one embodiment, the structure may be micelles, which are the simplest microemulsion structures that are generally spherical or cylindrical objects. Micelles are like drops of oil in water, and reverse micelles are like drops of water in oil. In an alternative embodiment, the microemulsion structure is the lamellae. It comprises consecutive layers of water and oil separated by layers of surfactant. The “oil” of microemulsions optimally comprises phospholipids. Any of the phospholipids detailed above for liposomes are suitable for embodiments directed to microemulsions. An active ingredient of the disclosure may be encapsulated in a microemulsion by any method generally known in the art.

In yet another embodiment, an active ingredient of the disclosure may be delivered in a dendritic macromolecule, or a dendrimer. Generally speaking, a dendrimer is a branched tree-like molecule, in which each branch is an interlinked chain of molecules that divides into two new branches (molecules) after a certain length. This branching continues until the branches (molecules) become so densely packed that the canopy forms a globe. Generally, the properties of dendrimers are determined by the functional groups at their surface. For example, hydrophilic end groups, such as carboxyl groups, would typically make a water-soluble dendrimer. Alternatively, phospholipids may be incorporated in the surface of a dendrimer to facilitate absorption across the skin. Any of the phospholipids detailed for use in liposome embodiments are suitable for use in dendrimer embodiments. Any method generally known in the art may be utilized to make dendrimers and to encapsulate an active ingredient of the disclosure therein. For example, dendrimers may be produced by an iterative sequence of reaction steps, in which each additional iteration leads to a higher order dendrimer. Consequently, they have a regular, highly branched 3D structure, with nearly uniform size and shape. Furthermore, the final size of a dendrimer is typically controlled by the number of iterative steps used during synthesis. A variety of dendrimer sizes are suitable for use in the disclosure. Generally, the size of dendrimers may range from about 1 nm to about 100 nm.

### II. Methods

In an aspect, the disclosure provides a method for treating atherosclerosis in a subject in need thereof. In another aspect, the disclosure provides a method for treating liver steatosis (also referred to herein as “hepatosteatosis”) in a subject in need thereof. In still another aspect, the disclosure provides a method for treating atherosclerosis and liver steatosis, simultaneously, in a subject in need thereof. In still yet another aspect, the disclosure provides a method for treating atherosclerosis in a subject undergoing dialysis. In certain embodiments, the subject undergoing dialysis may have renal disease or renal failure. The methods of treatment involve administering a therapeutically effective amount of a composition comprising trehalose, and optionally a trehalase inhibitor, to the subject. Administration of trehalose, and optionally a trehalase inhibitor, may be carried out using any of the compositions, modes of administration, and dosage forms described herein. As used herein, the terms “treating” and “treatment” refer to reduction in severity and/or frequency of signs or symptoms, elimination of signs or symptoms and/or underlying cause, prevention of the occurrence of symptoms and/or their underlying cause (e.g., prophylactic therapy), and improvement or remediation of damage. The treatment of atherosclerosis may be measured by a reduction in atherosclerotic lesion area. The lesion area may be reduced by 2% or more. For example, the lesion area may be reduced by about 2%, about 5%, about 10%, about 15%, about 20%, about 25%, about 30%, about 35%, about 40%, about 45%, about 50%, about 60%, about 70%, about 80%, about 90% or about 100%. The treatment of liver steatosis may be measured by a reduction in fat mass or a reduction in liver triglycerides. For example, the fat mass may be reduced by about 2%, about 5%, about 10%, about 15%, about 20%, about 25%, about 30%, about 35%, about 40%, about 45%, about 50%, about 60%, about 70%, about 80%, about 90% or about 100%. Additionally, the liver triglycerides may be reduced by about 2%, about 5%, about 10%, about 15%, about 20%, about 25%, about 30%, about 35%, about 40%, about 45%, about 50%, about 60%, about 70%, about 80%, about 90% or about 100%.

The compositions described herein are useful in the treatment of the signs and symptoms of atherosclerosis. Additionally, the compositions described herein are useful in the treatment of the signs and symptoms of liver steatosis. Further, the compositions described herein are useful in the treatment of the signs and symptoms of liver steatosis and atherosclerosis, simultaneously. Treatment of the signs of symptoms of atheroscerlosis and/or liver steatosis may result in a reduction in the severity and/or frequency of signs or symptoms, or may result in the elimination of signs or symptoms. Signs and symptoms of atherosclerosis may include high blood pressure, kidney failure, pain on exertion (in the chest or legs), chest pain or pressure (angina), sudden numbness or weakness in the arms or legs, difficult speaking or slurred speech, and drooping muscles in the face. Signs and symptoms of liver steatosis may include fatigue, vague abdominal discomfort, slightly enlarged liver, poor appetite, weight loss, weakness and confusion.

In addition to atherosclerosis and liver steatosis, a composition of the disclosure may also be useful in the treatment of insulin resistance, diabetes and obesity. For example, a composition of the disclosure may be useful in the treatment of liver steatosis and obesity. Additionally, a composition of the disclosure may be useful in the treatment of liver steatosis and diabetes. Further, a composition of the disclosure may be useful in the treatment of liver steatosis and insulin resistance.

Suitable subjects include, but are not limited to, a human, a livestock animal, a companion animal, a lab animal, and a zoological animal. In one embodiment, the subject may be a rodent, e.g. a mouse, a rat, a guinea pig, etc. In another embodiment, the subject may be a livestock animal. Non-limiting examples of suitable livestock animals may include pigs, cows, horses, goats, sheep, llamas and alpacas. In yet another embodiment, the subject may be a companion animal. Non-limiting examples of companion animals may include pets such as dogs, cats, rabbits, and birds. In yet another embodiment, the subject may be a zoological animal. As used herein, a “zoological animal” refers to an animal that may be found in a zoo. Such animals may include non-human primates, large cats, wolves, and bears. In preferred embodiments, the animal is a laboratory animal. Non-limiting examples of a laboratory animal may include rodents, canines, felines, and non-human primates. In certain embodiments, the animal is a rodent. In a preferred embodiment, the subject is human.

A subject may or may not be having a sign or symptom associated with atherosclerosis and/or liver steatosis. A skilled artisan will appreciate that pathological atherosclerosis and/or liver steatosis likely commences prior to diagnosis or the onset of symptoms associated with atherosclerosis and/or liver steatosis. In some embodiments, a subject is having a symptom associated with atherosclerosis and/or liver steatosis. In other embodiments, a subject is not having a symptom associated with atherosclerosis and/or liver steatosis. In still other embodiments, a subject has detectable atherosclerosis and/or liver steatosis but is not having any other symptom associated with atherosclerosis and/or liver steatosis. In yet still other embodiments, a subject has received treatment for atherosclerosis and/or liver steatosis.

### (a) Administration

For therapeutic applications, a therapeutically effective amount of a composition of the disclosure is administered to a subject. A “therapeutically effective amount” is an amount of trehalose sufficient to produce a measurable response (e.g., treatment of atherosclerosis and/or steatosis, reduction in signs or symptoms associated with atherosclerosis and/or steatosis). Actual dosage levels of active ingredients in a therapeutic composition of the disclosure can be varied so as to administer an amount of the active ingredient(s) that is effective to achieve the desired therapeutic response for a particular subject. The selected dosage level will depend upon a variety of factors including the activity of the therapeutic composition, formulation, the route of administration, the presence of a trehalase inhibitor, combination with other drugs or treatments, disease and longevity, and the physical condition and prior medical history of the subject being treated. In some embodiments, a minimal dose is administered, and dose is escalated in the absence of dose-limiting toxicity. Determination and adjustment of a therapeutically effective dose, as well as evaluation of when and how to make such adjustments, are known to those of ordinary skill in the art of medicine.

Toxicity and therapeutic efficacy of the compositions described herein can be determined by standard pharmaceutical procedures in cell cultures or experimental animals, e.g., procedures used for determining the maximum tolerated dose (MTD), the ED₅₀, which is the effective dose to achieve 50% of maximal response, and the therapeutic index (TI), which is the ratio of the MTD to the ED₅₀. Obviously, compositions with high TIs are the most preferred compositions herein, and preferred dosage regimens are those that maintain plasma levels of the trehalose at or above a minimum concentration to maintain the desired therapeutic effect. Dosage will, of course, also depend on a number of factors, the site of intended delivery, the route of administration, frequency of administration, and other pertinent factors known to the prescribing physician. The dosage range may be from each of 10, 20, 50, 75, 100, 150, 200, 300 mg/Kg body weight per day up to each of 400, 450, 500, 550, 600, 650, 700, 750, 800, 850, 900, 950 and 1000 mg/Kg body weight per day. Generally, however, dosage will be in the range of approximately 0.1 grams/kg/day to 1 g/kg/day. Preferably the dose is less than 0.54 grams/kg % day.

In some embodiments the trehalose is administered such that the total daily dose (on a day of administration) is between about 5 grams to 50 grams. In preferred embodiments the total per administration dose of trehalose is 8, 15 or 30 grams. In particular embodiments the trehalose is administered as a single dose of 5, 8, 15, 30, 40 or 50 grams.

In certain aspects, the dosing regimen is equal doses. In other aspects, gradually increasing doses, or gradually decreasing doses may be used. For example, in certain aspects, a subsequent dose may be greater or lesser than a prior dose by about 10%, 20%, 30%, 40%, 50%, or about 100%.

Administration is accomplished such that that the maximum endotoxin level is less than 5 EU per kilogram of body weight per hour. In particular aspects, the endotoxin level is less than about 1, 2, 3, or less than about 4 endotoxin units per kilogram of body weight per hour.

The frequency of dosing may be once, twice, three times or more daily or once, twice, three times or more per week or per month, as needed as to effectively treat the symptoms or disease. In certain embodiments, the frequency of dosing may be once, twice or three times daily. For example, a dose may be administered every 24 hours, every 12 hours, or every 8 hours. In a specific embodiment, the frequency of dosing may be three times per week. In another specific embodiment, the frequency of dosing may be once a week. In still another specific embodiment, the frequency of dosing may be daily.

Duration of treatment could range from a single dose administered on a one-time basis to a life-long course of therapeutic treatments. The duration of treatment can and will vary depending on the subject and the disease to be treated. For example, the duration of treatment may be for 1 day, 2 days, 3 days, 4 days, 5 days, 6 days, or 7 days. Or, the duration of treatment may be for 1 week, 2 weeks, 3 weeks, 4 weeks, 5 weeks or 6 weeks. Alternatively, the duration of treatment may be for 1 month, 2 months, 3 months, 4 months, 5 months, 6 months, 7 months, 8 months, 9 months, 10 months, 11 months, or 12 months. In still another embodiment, the duration of treatment may be for 1 year, 2 years, 3 years, 4 years, 5 years, or greater than 5 years. It is also contemplated that administration may be frequent for a period of time and then administration may be spaced out for a period of time. For example, duration of treatment may be 5 days, then no treatment for 9 days, then treatment for 5 days.

The timing of administration of the treatment relative to the disease itself and duration of treatment will be determined by the circumstances surrounding the case. Treatment could begin immediately, such as at the time of diagnosis, or treatment could begin following other therapies. Treatment could begin in a hospital or clinic itself, or at a later time after discharge from the hospital or after being seen in an outpatient clinic. Additionally, treatment could begin following dialysis.

Administration of the compositions described herein may be carried out as part of a treatment regimen that may include multiple instances of administration of trehalose-containing compositions as well as administration of other pharmaceutically active compositions. Such a regimen may be designed as a method of treatment for atherosclerosis and/or steatosis, and/or as a method of long-term maintenance of the health of a patient after having been treated for atherosclerosis and/or steatosis (e.g., prevention). The treatment regimen may be designed as a method of treating a subject that is asymptomatic for atherosclerosis and/or steatosis. Such treatment regimen will delay the onset of atherosclerosis and/or steatosis symptoms in a subject. It will be appreciated that determination of appropriate treatment regimens is within the skill of practitioners in the art.

Administration is performed using standard effective techniques, including peripherally (i.e. not by administration into the central nervous system) or locally to the central nervous system. Peripheral administration includes but is not limited to subcutaneous, intradermal, intravenous, intramuscular, and intraperitoneal. Local administration, including directly into the central nervous system (CNS) includes but is not limited to via a lumbar, intraventricular or intraparenchymal catheter or using a surgically implanted controlled release formulation. In certain embodiments, a composition of the disclosure may be administered via an infusion (continuous or bolus). In other embodiments, a composition of the disclosure may be administered orally. In still other embodiments, a composition of the disclosure may be administered parenterally in combination with orally.

Pharmaceutical compositions for effective administration are deliberately designed to be appropriate for the selected mode of administration, and pharmaceutically acceptable excipients such as compatible dispersing agents, buffers, surfactants, preservatives, solubilizing agents, isotonicity agents, stabilizing agents and the like are used as appropriate. Remington's Pharmaceutical Sciences, Mack Publishing Co., Easton Pa., 16Ed ISBN: 0-912734-04-3, latest edition, incorporated herein by reference in its entirety, provides a compendium of formulation techniques as are generally known to practitioners. Effective peripheral systemic delivery by intravenous or intraperitoneal or intramuscular or subcutaneous injection is a preferred method of administration in the absence of a trehalase inhibitor. Suitable vehicles for such injections are straightforward.

Over 99.5% of the trehalose is not absorbed into the blood stream. In addition, oral amounts of trehalose higher than 50 g a day in humans frequently cause diarrhea, bloating and discomfort. Thus, in particular aspects, the trehalose may be administered intravenously as an aqueous formulation to address poor absorption into the bloodstream and minimize undesirable metabolic events. In specific embodiments, the pH of the formulation is about 4.5 to 7.0, the osmolality of the formulation is about 280-330 mOsm/kg, the formulation contains less than 1.0, 0.9, 0.8, 0.7, 0.6, 0.5, 0.4, 0.3, 0.2, 0.1 or less endotoxin units per mL and the aqueous formulation is about 50%, 40%, 30%, 20%, 10%, 5% or less trehalose (w/v). The trehalose may be delivered over a suitable period. In some embodiments administration is complete within from about 75 to about 120 minutes, specifically within less than 90 minutes. Alternatively, when the trehalose is formulated in combination with a trehalase inhibitor, the trehalose may be administered orally.

In certain embodiments, effective serum levels of trehalose are achieved within from about 10 to about 20 or 30 or 40 or 50 or 60 minutes following trehalose administration. In certain embodiments, effective serum levels of the active ingredient are achieved within from about 5 to about 20 or 30 or 40 or 50 or 60 minutes following trehalose administration. In certain embodiments, effective serum levels of the active ingredient are achieved within from about 20 to about 20 or 30 or 40 or 50 or 60 minutes following trehalose administration. In certain embodiments, effective serum levels of the active ingredient are achieved within about 5, 10, 15, 20, 30, 40, 50 or 60 minutes following trehalose administration.

Further, methods of the disclosure may be used in combination with standard treatments for atherosclerosis or liver steatosis. Non-limiting examples of standard treatment include stress reduction, diet changes, lifestyle changes, drugs and surgery. Non-limiting examples of lifestyle changes include cessation of smoking, exercising, alcohol in moderation, and relaxation techniques such as mediation, progressive relaxation, yoga and biofeedback training. Non-limiting examples of diet changes include lowering sodium and trans fat consumption and increasing intake of fresh fruits and vegetables, whole unprocessed high-fiber grains, and healthy sources of fats and proteins. Non-limiting examples of drugs include aspirin, ACE inhibitors, angiotensin II receptor blockers, antiarrhythmics, beta-blockers, high blood pressure medication, high cholesterol medication, diuretics, water pills, calcium channel blocker drugs, clot buster drugs, digoxin, nitrates, antiplatelet drugs, blood thinners, and corticosteroids. Non-limiting examples of surgery include balloon angioplasty and stents, balloon valvuloplasty, heart bypass surgery, open heart surgery, pacemaker or defibrillator implantation, heart transplantation, cardioconversion, EECP, ablation, lead extraction, and left ventricular assist device (LVAD).

### EXAMPLES

The following examples are included to demonstrate preferred embodiments of the invention. It should be appreciated by those of skill in the art that the techniques disclosed in the examples that follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1. Trehalose Reduces the Size of Atherosclerotic Lesions

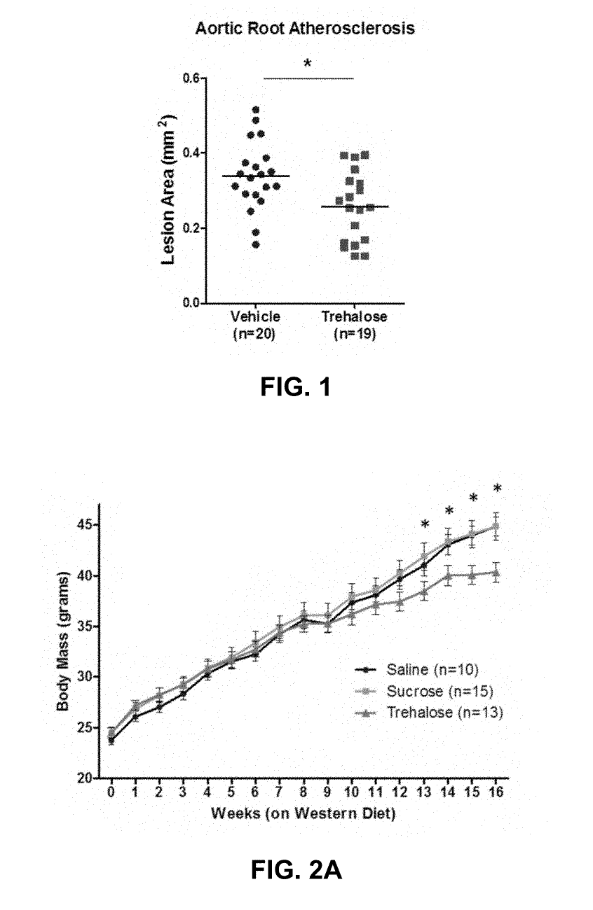

ApoE deficient mice were fed a Western diet with and without treatment with trehalose. Trehalose was administered oral and IP. At 2 months post-treatment the presence of atherosclerosis was evaluated. Trehalose significantly reduced atherosclerotic lesion size relative to vehicle treated animals (FIG. 1).

### Example 2. Trehalose Reduces the Presence of Steatosis

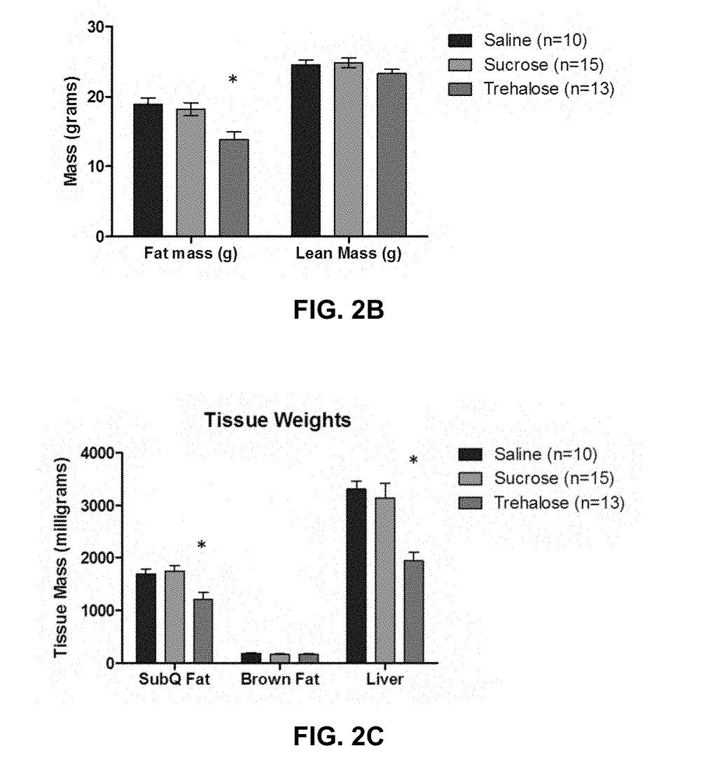

Mice were fed a Western diet in combination with saline, sucrose or trehalose. The saline, sucrose or trehalose was administered orally and IP. Oral administration was provided in the cage water supply at 2% w/v in water (w/v=weight/volume). Additionally, 2 grams trehlose/kg mouse weight was injected IP. IP injection was 3 times/week. At 13-16 weeks, mice treated with trehalose had significantly decreased body mass (FIG. 2A). It was then determined if this significant reduction in body mass was due to fat mass or lean mass. Mice evaluated at 16 weeks showed a significant reduction in fat mass upon treatment with trehalose relative to saline or sucrose. (FIG. 2B). It was then determined the location of the increase in fat mass via tissue weights. Mice treated with trehalose had a significant reduction in tissue mass in the liver suggesting that trehalose was reducing the presence of fat in the liver (FIG. 2C). Additionally, there was a reduction of fat mass in the subcutaneous (subQ) fat suggesting that trehalose also reduced the presence of fat in the adipose tissue. Finally, the amount of liver triglycerides was measured in each group. There was a significant reduction in liver triglycerides in the trehalose treated animals confirming that the reduction in liver mass was due to a reduction in the presence of fat in the liver (FIG. 2D).

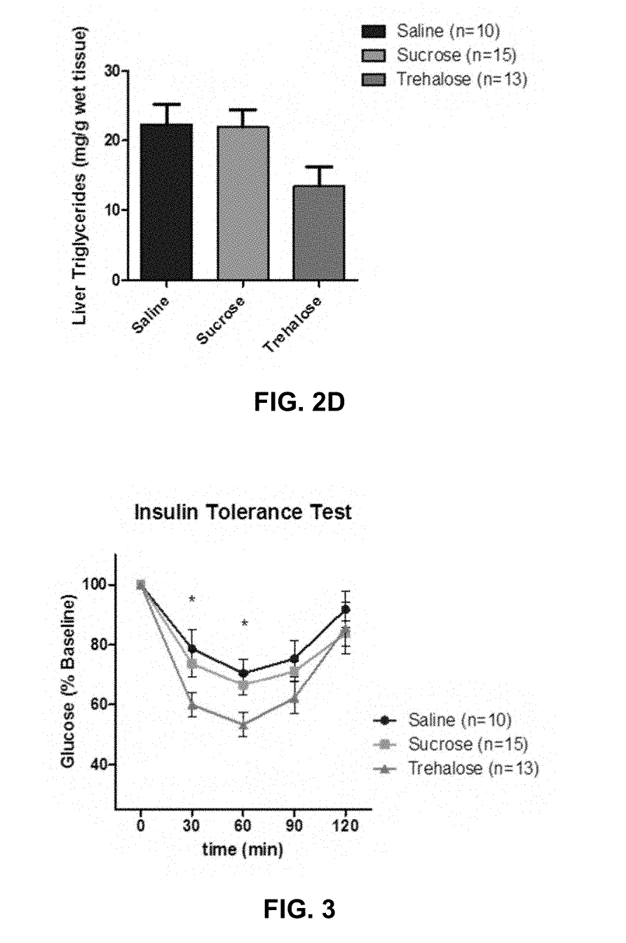

Additionally, an insulin tolerance test was performed in the presence of saline, sucrose or trehalose. Results showed that less insulin resistance was observed after trehalose administration (FIG. 3).

### Example 3. Trehalose Activity in the Presence of a Trehalase Inhibitor

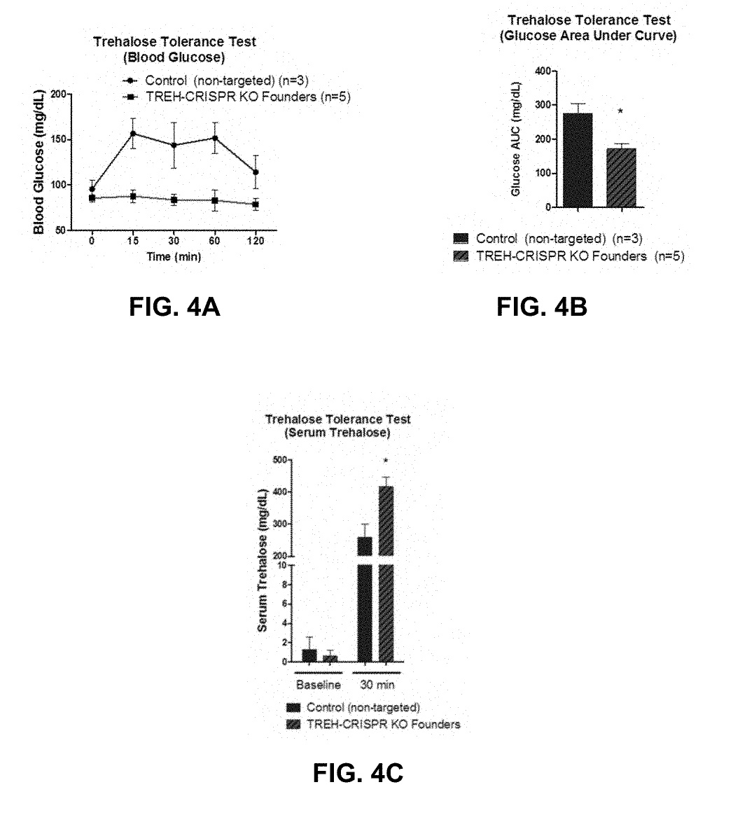

To determine if inhibition of trehalase could enhance the efficacy of trehalose, a trehalase knockout mouse was generated. Control mice and trehalase knockout mice were then administered trehalose IP at 1 g/kg. Of 12 chimeric founders, a trehalose tolerance test was performed on n=3 non-targeted controls and n=5 chimeric mice. FIG. 4A shows the blood glucose levels at indicated times and FIG. 4B shows the glucose area under curve (AUC). The serum concentration of trehalose at 30 minutes post-administration was measured. Mice with an inactive trehalase achieved significantly higher serum levels of trehalose at 30 minutes (FIG. 4C). This data suggested that inhibition of trehalase is a valid means to improve the bioavailability of trehalose.

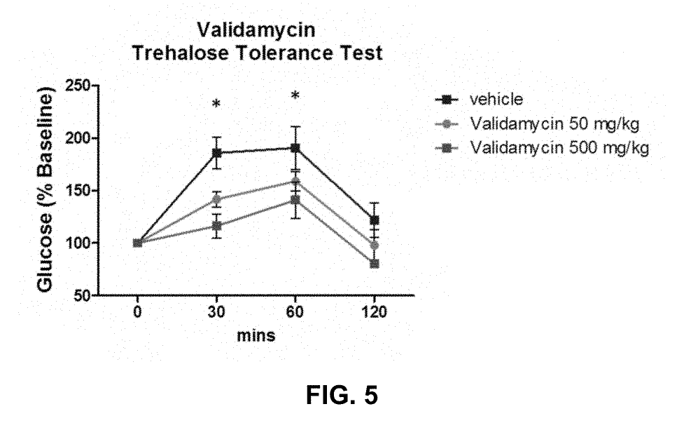

Validamycin is a known trehalase inhibitor. To confirm the results observed in the trehalase knockout mice, the activity of trehalose in the presence of validamycin was evaluated. Mice were administered trehalose and administered validamycin at 50 mg/kg or 500 mg/kg. The activity of trehalose was measured via blood glucose levels. Mice co-administered the trehalose and validamycin exhibited a significant reduction in blood glucose levels (FIG. 5). These results indicate that the presence of a trehalase inhibitor improves the activity of trehalose.

1. A method for treating atherosclerosis in a subject in need thereof, the method comprising administering a therapeutically effective amount of a composition comprising trehalose, and optionally a trehalase inhibitor, to the subject.

2.-4. (canceled)

5. The method of claim 1, wherein the composition is administered orally or intravenously, wherein when the composition is administered orally, the trehalase inhibitor is present and when the composition is administered intravenously, the trehalase inhibitor is absent.

6.-7. (canceled)

8. The method of claim 1, wherein the trehalase inhibitor is validamycin.

9.-12. (canceled)

13. The method of claim 1, wherein the trehalose is administered at a dose of 5 to 50 grams per administration.

14. (canceled)

15. The method of claim 1, wherein the trehalose is administered at 8, 15 or 30 grams per day.

16. (canceled)

17. The method of claim 1, wherein treatment of atherosclerosis is measured by a reduction in atherosclerotic lesion area or a reduction in the signs and symptoms associated with atherosclerosis.

18.-20. (canceled)

21. The method of claim 1, wherein the subject is undergoing dialysis, wherein the composition is administered 1, 2, or 3 times per week following dialysis.

22. A method for treating liver steatosis in a subject in need thereof, the method comprising administering a therapeutically effective amount of a composition comprising trehalose, and optionally a trehalase inhibitor, to the subject.

23. The method of claim 22, wherein the composition is administered orally or intravenously, wherein when the composition is administered orally, the trehalase inhibitor is present and when the composition is administered intravenously, the trehalase inhibitor is absent.

24. The method of any of claim 22, wherein the trehalase inhibitor is validamycin.

25. The method of claim 22, wherein the trehalose is administered at a dose of 5 to 50 grams per administration.

26. The method of claim 22, wherein the trehalose is administered at 8, 15 or 30 grams per day.

27. The method of claim 22, wherein treatment of liver steatosis is a reduction in the signs and symptoms associated with liver steatosis or is measured by a reduction in fat mass or liver triglycerides.

28. The method of claim 22, wherein insulin resistance, obesity and/or diabetes is also treated.

29. A method for treating atherosclerosis and liver steatosis, simultaneously, in a subject in need thereof, the method comprising administering a therapeutically effective amount of a composition comprising trehalose, and optionally a trehalase inhibitor, to the subject.

30. The method of claim 29, wherein the composition is administered orally or intravenously, wherein when the composition is administered orally, the trehalase inhibitor is present and when the composition is administered intravenously, the trehalase inhibitor is absent.

31. The method of claim 29, wherein the trehalase inhibitor is validamycin.

32. The method of claim 29, wherein the trehalose is administered at a dose of 5 to 50 grams per administration.

33. The method of claim 29, wherein the trehalose is administered at 8, 15 or 30 grams per day.

34. The method of claim 29, wherein treatment of atherosclerosis is measured by a reduction in atherosclerotic lesion area or a reduction in the signs and symptoms associated with atherosclerosis.
